(19) United States
(12) Patent Application Publication
(10) Pub. No.: US 2008/0138447 A1
(43) Pub. Date: Jun. 12, 2008

(54) Method for administering appetite suppressant and composition thereof

(76) Inventors: Erin John Riggins, Marietta, GA (US); Merritt Maynard Ambrose, Alpharetta, GA (US)

Correspondence Address: SEYFARTH SHAW LLP, 131 S. DEARBORN ST., SUITE 2400, CHICAGO, IL, 60603-5803, US

(21) Appl. No.: 11/634,423

(22) Filed: Dec. 6, 2006

(51) Int. Cl. A61K 36/25

(52) U.S. Cl. 424/728

(57) ABSTRACT

A composition, which includes Guarana extract 36%, Hoodia 20:1, Cha de bugre 4:1, Ginseng, Citrus Auratium, Magnolia bark extract, and black pepper, for suppressing appetite as well as a method for the weight reduction of a body by administering a dosage of the composition, are disclosed. The composition, which is preferably a 575 mg dosage, contains an amount of Guarana extract 36% within the range of from about 40% to about 70% by weight. The composition contains an amount of Hoodia 20:1 within the range of from about 12% to about 30% by weight. The composition contains an amount of Cha de bugre 4:1 within the range of from about 5% to about 25% by weight. The composition contains an amount of Ginseng within the range of from about 2.0% to about 8.0% by weight. The composition contains an amount of Citrus Auratium within the range of from about 2.0% to about 8.0% by weight. The composition contains an amount of Magnolia bark extract within the range of from about 2.0% to about 8.0% by weight. The composition contains an amount of black pepper within the range of from about 2.0% to about 8.0% by weight.

### TECHNICAL FIELD OF THE INVENTION

The present method and composition relate to appetite suppressants. Particularly, the present composition and method relate to an all-natural appetite suppressant.

### BACKGROUND OF THE INVENTION

Whether trying to shed a few extra pounds or the effects of a lifetime of over-indulging and poor eating habits, diet and exercise are key elements of any successful program. However, some people can be easily discouraged by lapses or a lack of self-discipline making long-term weight loss changes a seemingly insurmountable burden.

Accordingly, many dieters have turned to using nutritional supplements to help stay hunger pangs and increase their chances of long-term success. One relatively recent entry to the field is known as Hoodia Gordonii.

Hoodia, pronounced “who-DEE-ah,” Gordonii is a leafless spiny succulent plant with fleshy finger like stems and is also known as “Xhoba.” Technically speaking, it is different from a cactus. Hoodia Gordonii grows naturally in the harsh desert conditions of the Kalahari Desert of Southern Africa. It has also been cultivated in semi-arid areas of China, Mexico and the United States with limited success. The plant grows to a mature height of six-feet and may survive for as much as a century or more. The Hoodia plant belongs in the succulent family of Asclepiadaceous, along with about 20 other species including statelier, stephanotis and vinca.

As Hoodia grows, it forms multi-stemmed clumps 12 inches wide by 12 inches high and bears 3-4 inch diameter unpleasant-smelling, pale, purple, saucer-shaped flowers. Row of thorns are present along the stems.

It is said that the San people of the Kalahari have learned to eat the bitter-tasting plant to suppress their appetite and thirst when on long hunting expeditions. By taking the edge off appetite and thirst, the hunters are able to respect their tradition of bringing home their entire catch without eating of it on the return trip.

Hoodia is registered as a protected species by the South African Nature Conservation and farmers are granted a special permit allowing Hoodia to be commercially grown and harvested for sale to the consumer market. Hoodia takes two years to grow and is now being grown in sustainable quantities by South African farmers. It has become widely known as one of the greatest appetite suppressant of all time.

### SUMMARY OF THE INVENTION

There is disclosed herein an improved appetite suppressant which avoids the disadvantages of prior compositions and methods while affording additional benefits and advantages.

In one embodiment of a composition for suppressing appetite, the invention includes Guarana extract 36%, Hoodia 20:1, Cha de Bugre 4:1, Ginseng, Citrus Auratium, Magnolia bark extract, and black pepper. The composition contains an amount of Guarana extract 36% within the range of from about 40% to about 70% by weight. The composition contains an amount of Hoodia 20:1 within the range of from about 12% to about 30% by weight. The composition contains an amount of Cha de Bugre 4:1 within the range of from about 5% to about 25% by weight. The composition contains an amount of Ginseng within the range of from about 2.0% to about 8.0% by weight. The composition contains an amount of Citrus Auratium within the range of from about 2.0% to about 8.0% by weight. The composition contains an amount of Magnolia bark extract within the range of from about 2.0% to about 8.0% by weight. The composition contains an amount of black pepper within the range of from about 2.0% to about 8.0% by weight.

In a disclosed method for body weight reduction, an embodiment of the invention includes orally administering to a person a dosage comprising Hoodia 20:1 in an amount within the range of from about 69 to about 170 mg. The dosage may include any or all of Guarana extract, in an amount within the range of from about 230 to about 405 mg, Cha de Bugre 4:1, in an amount within the range of from about 28 to about 145 mg, and Ginseng, Citrus Auratium, Magnolia bark extract, and black pepper, each in an amount within the range of from about 11 to about 46 mg. The total dosage is within the range of from about 300 to about 700 mg, preferably about 575 mg.

These and other embodiments of the invention may be understood more readily from the following detailed description.

### DETAILED DESCRIPTION OF PREFERRED EMBODIMENTS

While this invention is susceptible of embodiments in many different forms, there is described in detail herein a preferred embodiment of the invention with the understanding that the present disclosure is to be considered as an exemplification of the principles of the invention and is not intended to limit the broad aspect of the invention to embodiments described.

The disclosed composition contains an effective amount of Hoodia Gordonii combined with several other ingredients. It is believed that the combination of these additional ingredients creates a synergy in the efficacy of the Hoodia. That is, the additional ingredients are believed to permit the use of less Hoodia in the total composition to achieve the same appetite suppressant success as that of a larger dosage of pure Hoodia.

The additional ingredients used in the embodiments of the present composition are as follows:

### Guarana Extract

Guarana comes from the Amazon rainforest and is used to improve mental sharpness, reduce fatigue, increase stamina and endurance, and may even lift depressed spirits.

Guarana is a shrubby, climbing vine that is native to South America, with compound leaves, yellow-flowered panicles, and pear-shaped fruit, filled with seeds like a small horse chestnut. It thrives in moist, humus-rich soil in the partial shade of the rainforest at a minimum temperature of sixty-five degrees Fahrenheit. Guarana is made by the Guaranis, a tribe of South American Indians, in a long and complicated process. The seeds are shelled, washed, roasted, and pounded into a fine powder that is mixed with water to make dough.

The dough is then rolled and sun-dried (or over a slow fire) until it hardens and is cut into cylindrical pieces. The result is a bitter, chocolate-flavored substance (without chocolate's oiliness) that is used in refreshing teas or as a tasty, coffee-like drink that is said to increase energy and mental acuity, combat fatigue and promote endurance and stamina. Guarana is said to be the richest source of caffeine worldwide, well-known for its stimulating effects due to that high caffeine content.

Some of the constituents in Guarana include adenine, caffeine, D-catechin, saponin, tannins, theobromine, theophylline, starch and a fixed oil, and a crystallizable principle in the seeds called guaranine.

Guarana is a stimulating tonic that is believed to improve physical stamina and endurance. Because guaranine is almost identical to caffeine in its actions, athletes have been known to take it to stimulate and improve their performance and increase their strength and endurance. It is also thought to reduce fatigue and exhaustion. The combination of guarinine, theobromine and theophyline act to stimulate the central nervous system and also act to enhance the metabolic rate, which may be helpful in weight loss programs.

As a “nervine,” Guarana is also said to strengthen functional activity of the nervous system. The alkaloids theobromine, and theophylline act to stimulate the central nervous system and are thought to be useful in cases of neuralgia, paralysis, migraine and nervous headache and the distress that sometimes accompanies menstruation.

Guarana is considered a gentle excitant that is said to be good for depression and mental exertion where there is fatigue or even exhaustion from hot weather. It is also thought to increase mental sharpness and concentration, which may also be a result of its guarinine content.

The tannins in Guarana act as an astringent and are said to help ease mild forms of leucorrhoea (vaginal discharge) and diarrhea. As a mild diuretic, Guarana is thought to promote urine flow and act as febrifuge that helps to reduce mild fevers. It is also said to alleviate urinary tract irritation.

### Cha De Bugre

Chá de bugre (Cordia salicifolia—Brazil, Cordia ecalyculata-Paraguay) is a small tree growing 8-12 meters in height with a trunk 30-40 cm in diameter. It is indigenous to Brazil and can be found growing predominately in the Brazilian states of Minas Gerais, Bahia, Acre and Goias. It is also found in tropical forest areas of Argentina and Paraguay. In Brazil, it is commonly called café do mato (coffee of the woods) because it produces a red fruit resembling a coffee bean which is roasted and brewed into tea as a coffee substitute.

Chá de bugre products are highly commercialized as a weight loss aid in Brazil where tea bags, fluid extracts and tinctures of chá de bugre are commonly seen in pharmacies, stores, and even in the beach-front eateries and refreshment stands along Rio de Janeiro's beaches. It has long been a popular weight loss product which has been marketed as a diuretic, appetite suppressant, and believed to help prevent or reduce fatty deposits and cellulite. Dr. C. L. Cruz in his book, Dictionary of the Plants Used in Brazil, recommends chá de bugre as an excellent diuretic and weight loss aid as well as a good general heart tonic which can help stimulate circulation. It is also used in Brazil and Haiti as a tea to help relieve coughs, regulate renal function, reduce uric acid and, used externally, to heal wounds.

Despite the popularity of chá de bugre in Brazil very little has been done to analyze the phytochemicals in the plant. At present it is known to contain caffeine, potassium, allantoin and allantoic acid. The red fruits or berries of chá de bugre (resembling a coffee bean) contain caffeine. The allantoin and allantoic acid may explain the traditional use of the plant for wound healing. Main plant chemicals include allantoin, allantoic acid, caffeine, potassium.

Very little clinical research is thought to exist on chá de bugre. However, some possible uses for chá de bugre include reducing herpes virus penetration, inhibition of cancer cells, and a as a heart tonic.

As an appetite suppressant, rather than cutting off appetite all together (then causing intense hunger when it wears off at the wrong time) chá de bugre gives one a sense of being full and satiated after eating only a few bites of food. This seems to promote much smaller meals, more often, which is what many practitioners believe is better for sustained weight loss and keeping the metabolism going throughout the day.

### Ginseng

The use of Ginseng is thought to be an excellent way to improve the body's resistance to infection and damaging environmental influences. It is also used by many athletes for overall body strengthening and endurance. Ginseng has been used for treatment of bronchitis, circulatory problems, diabetes and infertility. Some believe it may even be helpful in lowering cholesterol and possibly even inhibiting the growth of tumors. It has long been used as an aphrodisiac and is especially helpful to weak or elderly people.

American Ginseng is a smaller version of its more famous Asian (Korean/Chinese) cousin but has many of the same benefits. It is a slow-growing perennial plant with a large fleshy root (the part used in herbal medicine) and a stem that grows to two feet. It is found from Maine to Georgia and from Oklahoma to Minnesota, and it is endangered in much of this area.

Generally speaking, Ginseng normalizes body functions during stressful situations which tend to alter those functions. This “normalization” helps the body to adapt and return to an overall sense of well-being. It also improves mental and physical vigor and is used by athletes for overall body strengthening and endurance.

Ginseng helps to combat stress because it appears to protect a portion of the brain known as the hippocampus from the effects of stress hormones. This prevents memory problems and loss of cognitive ability in people who suffer from bipolar disorder and even depression. It may be used to relieve fatigue, stress and nervousness, especially after acute illness.

Ginseng is believed to promote a good appetite, stimulate fertility in women, and is helpful for rheumatism, headaches, colds, coughs, bronchitis, constipation, cystitis and symptoms of menopause. It has anti-inflammatory properties which may be useful in reducing fevers and lung problems. Taken in low doses, it acts as a mild sedative; in large doses, it acts as a stimulant.

It has been used to reduce cholesterol, high blood pressure, and may be useful to inhibit the growth of cancerous tumors. Researchers also believe that it may be a viable alternative to conventional forms of treatment for Type-2 diabetes.

Ginseng is said to increase vitality and improve the body's resistance to a wide variety of illnesses and damaging external influences. It strengthens the adrenal and reproductive glands, enhances immune functions and promotes lung and respiratory health.

### Citrus Aurantium

Citrus Aurantium, a fruit commonly known as bitter orange, has been used in traditional Chinese medicine to treat chest congestion and indigestion, stimulate gastrointestinal function and improve circulation and liver function. In traditional western medicine, Citrus Aurantium has been used to treat digestive and circulatory problems.

Citrus Aurantium is widely used for stimulating the breakdown of fat, by causing the release of noradrenaline (a stress hormone) at beta-3 receptor sites creating chemical reactions that increase fat breakdown. Beta-3 receptors in the body increase the rate at which fat is released from the body stores (lipolysis) and increase resting metabolic rate (thermogenesis). Physical activity tends to increase this thermogenic effect and further enhances the thermogenic effect of Citrus Aurantium towards healthy and permanent weight loss.

### Magnolia Bark Extract

In Chinese medicine Magnolia Bark has been associated with the stomach, lungs, spleen, and large intestine for over two thousand years and has been used to treat abdominal bloating, gas, nausea, diarrhea, menstrual cramps, and indigestion. Recent studies have found that the herb inhibits the production of cortisol (the substance that encourages fat storage) and may be effective in weight loss programs.

Magnolia is a magnificent family of forest trees that are revered for their beautiful, large, showy, and fragrant flowers. Magnolias may be both evergreen and deciduous with luxuriant foliage and rich flowers and can reach a height of more than eighty feet, with some species much smaller. Magnolias can survive in both moist and dry soils that can be neutral-to-acid-to-alkaline, in sun or partial shade with shelter from cold winds and late frosts. When growing in warmer climates, the trees reach their greatest development.

Magnolia Bark is collected in the autumn, and the unopened flowers are harvested in the springtime and used in herbal medicines. Some of the constituents in Magnolia Bark include volatile oils (eudesmol, bornyl-acetate, etc.), alkaloids, tannin, magnolol, honokiol, zinc, copper, calcium, potassium, iron, magnesium, and manganese.

Magnolia Bark has been used in Chinese herbal medicine for at least two thousand years as an aromatic, pungent, and warming stimulant that treats various disorders of the digestive system and strengthens stomach function. It is a bitter relaxant herb that acts as a tonic and improves digestion; relieves stomach pains, gastroenteritis, and flatulence; calms diarrhea and vomiting associated with indigestion; stimulates poor appetite; and alleviates fullness and distension of the abdomen.

As a mild diaphoretic, Magnolia Bark is said to increase perspiration and sweating and thus reduce fevers and cool the body. It has been used in cases of malarial fevers and fevers of a typhoid type.

Magnolia Bark is believed to have antiseptic, antibacterial, antifungal, antispasmodic, expectorant, and anti-inflammatory properties. As such, the bark is thought to relieve the pain and inflammation of rheumatism; counteract yeast infections (such as leukhorrea); combat upper respiratory tract infections and spasms, such as asthma, coughs, profuse phlegm in the lungs, shortness of breath, and fullness and pressure in the chest area.

In the fight against obesity and weight management, Magnolia Bark has recently been recognized as an efficient fat burner. It is said that the magnolol and honokial in the herb effectively inhibit the body's production of cortisol, the substance that liberates fat from adipose cells and thus suppresses fat storage, particularly in the abdominal and belly area.

### Black Pepper

Black pepper has been used for years as a stimulant for taste. Use in the present composition is to give the user a warm and biting reaction to taking the capsule. It is not intended to produce dietary results, only the feeling that something good is happening. Obviously, other taste stimulators known by those skilled in the art may be used to provide the same or similar sensation.

Given these components, a dosage of between about 300 and 700 mg, preferably about 575 mg, using varied amounts of each within specified ranges can be composed to offer various synergistic effects. The formula ranges shown in TABLE 1 are general weight loss formulas for obtaining the benefits of each component while still avoiding the potential side effects of having too much of any one component.

TABLE 1
300–700 mg Dosage
575 mg Preferred Dosage

| | Percent | Weight | Preferred | Preferred |
|---|---|---|---|---|
| | (%) | (mg) | Percent (%) | Weight |
| COMPONENT | by Weight | Range | by Weight | (mg) |
| Hoodia 20:1 | 12–30 | 69–173 | 17.3 | 100 |
| Guarana Extract 36% | 40–70 | 230–405 | 47.8 | 275 |
| Cha De Bugre 4:1 | 5–25 | 28–145 | 17.3 | 100 |
| Ginseng | 2–8 | 11–46 | 4.3 | 25 |
| Citrus Aurantium | 2–8 | 11–46 | 4.3 | 25 |
| Magnolia Bark | 2–8 | 11–46 | 4.3 | 25 |
| Black Pepper | 2–8 | 11–46 | 4.3 | 25 |

Note that the “Preferred Percent (%) by Weight” column only totals 99.6%. The non-active ingredients used to prepare the dosage form, such as Magnesium stearate which is widely used as a lubricant in the manufacture of pharmaceutical solid dosage forms, have not been accounted for. Those skilled in the art would understand the use of such ingredients to produce the desired dosage form.

To suppress appetite, the preferred dosage of 575 mg should be administered to the individual twice daily, once in the morning (at least one hour before breakfast) and once in the afternoon (at least one hour before dinner) being the preferred dosage schedule. The total dosage may be within the range of from about 300 mg to about 700 mg, with the daily dosing being adjusted accordingly. That is, if using smaller dosages, e.g., 300 mg tablets, then in some cases it may be desirable to administer two tablets (i.e., 600 mg total) at each of the prescribed times.

The matter set forth in the foregoing description is offered by way of illustration only and not as a limitation. While particular embodiments have been described, it will be apparent to those skilled in the art that changes and modifications may be made without departing from the broader aspects of applicants' contribution. The actual scope of the protection sought is intended to be defined in the following claims when viewed in their proper perspective based on the prior art.

1. A composition for suppressing appetite comprising:
Guarana extract 36% in the range of from 40% to 70% by weight,
Hoodia 20:1 in the range of from 12% to 30% by weight,
Cha de bugre 4:1 in the range of from 5% to 25% by weight,
Ginseng in the range of from 20% to 8.0% by weight,
Citrus Auratium in the range of from 2.0% to 8.0% by weight,
Magnolia bark extract in the range of from 2.0% to 8.0% by wieght, and
black pepper in the range of from 2.0% to 8.0% by weight.

2. The composition of claim 1, wherein the amount of Guarana extract 36% is within the range of from about 45% to about 60% by weight.

3. The composition of claim 1, wherein the amount of Hoodia 20:1 is within the range of from about 15% to about 25% by weight.

4. The composition of claim 1, wherein the amount of Cha de Bugre 4:1 is within the range of from about 8% to 20% by weight.

5. The composition of claim 1, wherein the amount of Ginseng is about 5% by weight.

6. The composition of claim 1, wherein the amount of Citrus Auratium is about 5% by weight.

7. The composition of claim 1, wherein the amount of Magnolia bark extract is about 5% by weight.

8. The composition of claim 1, wherein the amount of black pepper is about 5% by weight.

9. A method for body weight reduction comprising the step of administering to a person a dosage comprising Hoodia 20:1 in an amount within the range of from about 69 to about 170 mg together with Guarana extract, in an amount within the range of from about 230 to about 405 mg, Cha de Bugre 4:1, in an amount within the range of from about 28 to about 145 mg, Ginseng, Citrus Auratium, Magnolia bark extract, and black pepper, each in an amount within the range of from about 11 to about 46 mg.

10. The method of claim 9, wherein the total dosage is about 575 mg.

11. The method of claim 9, wherein the total dosage is within the range of from about 300 to about 700 mg.

12. The method of claim 9, wherein the amount of Hoodia 20:1 is about 100 mg.

13. The method of claim 9, wherein the amount of Cha de bugre is about 100mg.

14. The method of claim 9, wherein the amount of Guarana extract is about 275 mg.

15. A method for body weight reduction comprising the step of administering to a person a total dosage of about 300 to 700 mg comprising an effective amount of Hoodia 20:1 together with Guarana extract, Cha de bugre 4:1, Ginseng, Citrus Auratium, Magnolia bark extract, and black pepper.

16. The method of claim 15, wherein the total dosage is about 575 mg.

17. The method of claim 15, wherein the amount of Hoodia 20:1 is about 100mg.

18. The method of claim 15, wherein the amount of Cha de bugre is about 100mg.

19. The method of claim 15, wherein the amount of Guarana extract is about 275 mg.
